(19) United States
(12) Patent Application Publication
(10) Pub. No.: US 2026/0345250 A1
(43) Pub. Date: Nov. 5, 2026

(54) INTRA-OPERATIVE SPATIO-TEMPORAL PREDICTION OF CRITICAL STRUCTURES

(71) Applicant: Covidien LP, Mansfield, MA (US)

(72) Inventors: Faisal I. Bashir, Andover, MA (US); Meir Rosenberg, Newton, MA (US)

(21) Appl. No.: 19/477,896

(22) PCT Filed: Apr. 10, 2024
(86) PCT No.: PCT/IB2024/053514

§ 371 Date: Oct. 23, 2025

Related U.S. Application Data
(60) Provisional application No. 63/461,630, filed on Apr. 25, 2023.

(51) Int. Cl. A61B 34/20; A61B 34/10; G06N 3/0499; G06N 3/0895; G06V 10/62; G06V 10/82; G06V 20/40

(52) U.S. Cl. CPC A61B 34/20; G06N 3/0499; G06N 3/0895; G06V 10/62; G06V 10/82; G06V 20/41; G06V 20/44; A61B 2034/105; A61B 2034/107; A61B 2034/2065; A61B 2034/2074

(57) ABSTRACT

Examples described herein provide a computer-implemented method that includes providing video frames of a video stream to an anticipative model trained for a surgical procedure. The method also includes providing a three-dimensional (3D) model of an anatomical feature of the patient to the anticipative model, determining a predicted future state of the surgical procedure based on a prediction output of the anticipative model, and outputting an indicator associated with the predicted future state.

### CROSS-REFERENCE TO RELATED APPLICATION

This application claims the benefit of U.S. Provisional Patent Application Ser. No. 63/461,630, filed Apr. 25, 2023, the entire content of which is incorporated herein by reference.

### BACKGROUND

Aspects relate in general to computing technology and more particularly to computing technology for intra-operative spatio-temporal prediction of critical structures.

Computer-assisted systems, particularly computer-assisted surgery systems (CASs), rely on video data digitally captured during a surgery. Such video data can be stored and/or streamed. In some cases, the video data can be used to augment a person's physical sensing, perception, and reaction capabilities. For example, such systems can effectively provide the information corresponding to an expanded field of vision, both temporal and spatial, that enables a person to adjust current and future actions based on the part of an environment not included in his or her physical field of view. Alternatively, or in addition, the video data can be stored and/or transmitted for several purposes, such as archival, training, post-surgery analysis, and/or patient consultation.

In various surgical procedures, certain anatomical structures may be classified as critical structures. Critical structures can be associated with establishing a critical view of safety before advancing to a next step, such as cutting or other surgical instrument use that may impact surgical outcome.

### SUMMARY

According to an aspect, a computer-implemented method is provided. The method includes receiving a video stream including a plurality of video frames of a surgical procedure and providing the video frames to an anticipative model trained for the surgical procedure. The method also includes providing a three-dimensional (3D) model of an anatomical feature of the patient to the anticipative model, determining a predicted future state of the surgical procedure based on a prediction output of the anticipative model, and outputting an indicator associated with the predicted future state.

According to another aspect, a system includes a machine learning training system configured to use a training dataset to train a 3D model of an anatomical structure and an anticipative model to predict a future state of a surgical procedure based on the training dataset and the 3D model. The system also includes a data collection system configured to capture a video of the surgical procedure and a model execution system configured to execute the anticipative model to determine a predicted future state of the surgical procedure based on a prediction output of the anticipative model with respect to the video. The system further includes a detector configured to generate an indicator associated with the predicted future state and output the indicator to a user interface.

According to a further aspect, a computer program product includes a memory device having computer executable instructions stored thereon, which when executed by one or more processors cause the one or more processors to perform a plurality of operations including receiving a video stream comprising a plurality of video frames of a surgical procedure, providing the video frames to an anticipative model trained for the surgical procedure, determining a predicted future state of the surgical procedure based on a prediction output of the anticipative model, and outputting an indicator associated with the predicted future state.

The above features and advantages, and other features and advantages, of the disclosure are readily apparent from the following detailed description when taken in connection with the accompanying drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a computer-assisted surgery (CAS) system according to one or more aspects;

FIG. 2 depicts a surgical procedure system according to one or more aspects;

FIG. 3 depicts a system for machine learning and model execution according to one or more aspects;

FIG. 4 depicts a time sequence diagram of future action predictions according to one or more aspects;

FIG. 5 depicts a block diagram of an anticipative video transformer network according to one or more aspects;

FIG. 6 depicts an image sequence of critical structure prediction according to one or more aspects;

FIG. 7 depicts an image sequence of temporal recognition of events according to one or more aspects;

FIG. 8 depicts a flowchart of a method of future state prediction of a surgical procedure according to one or more aspects;

FIG. 9 depicts a block diagram of a computer system according to one or more aspects; and

FIG. 10 depicts a system for predicting future states of surgical procedures according to one or more aspects.

The diagrams depicted herein are illustrative. There can be many variations to the diagrams and/or the operations described herein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of the technical solutions described herein include systems and methods for intra-operative spatio-temporal prediction of critical structures and/or other aspects of a surgical procedure. Predictions of future states can include location and appearance predictions at one or more future times during a surgical procedure. A three-dimensional (3D) model can be used in conjunction with a video stream to predict when structures will likely appear and a location of appearance. The 3D model can be a registered model constructed specifically for a patient using other scanning and imaging techniques to target a specific structure, such as a tumor.

Aspects, as further described herein, include a framework that can predict future states of a surgical procedure over various time horizons. Predictions can include multiple futures from which a most likely future may be selected. For example, future states may represent a median surgical outcome, an improved surgical outcome, and/or a less favorable surgical outcome. Tracking performance against predictions can be used to determine whether one future state is more likely than another in selecting a prediction output from a plurality of possible futures. For instance, if a surgical procedure has been predicted to proceed at a faster than median pace and transitions between phases have been observed to occur at a faster than median pace, then a predicted future event or phase that occurs earlier in time may be determined as more likely that other future states predicted at a median pace or a slower than median pace.

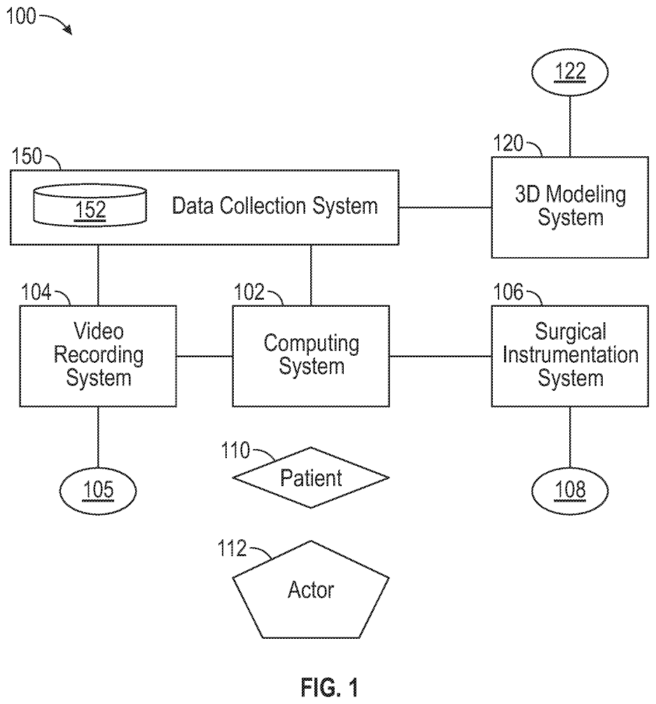

Turning now to FIG. 1, an example computer-assisted system (CAS) system 100 is generally shown in accordance with one or more aspects. The CAS system 100 includes at least a computing system 102, a video recording system 104, and a surgical instrumentation system 106. As illustrated in FIG. 1, an actor 112 can be medical personnel that uses the CAS system 100 to perform a surgical procedure on a patient 110. Medical personnel can be a surgeon, assistant, nurse, administrator, or any other actor that interacts with the CAS system 100 in a surgical environment. The surgical procedure can be any type of surgery, such as but not limited to cataract surgery, laparoscopic cholecystectomy, endoscopic endonasal transsphenoidal approach (eTSA) to resection of pituitary adenomas, or any other surgical procedure. In other examples, actor 112 can be a technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, and/or the like including combinations and/or multiples thereof.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions. In addition, a particular anatomical structure of the patient may be the target of the surgical action(s).

The video recording system 104 includes one or more cameras 105, such as operating room cameras, endoscopic cameras, and/or the like including combinations and/or multiples thereof. The cameras 105 capture video data of the surgical procedure being performed. The video recording system 104 includes one or more video capture devices that can include cameras 105 placed in the surgical room to capture events surrounding (i.e., outside) the patient being operated upon. The video recording system 104 further includes cameras 105 that are passed inside (e.g., endoscopic cameras) the patient 110 to capture endoscopic data. The endoscopic data provides video and images of the surgical procedure.

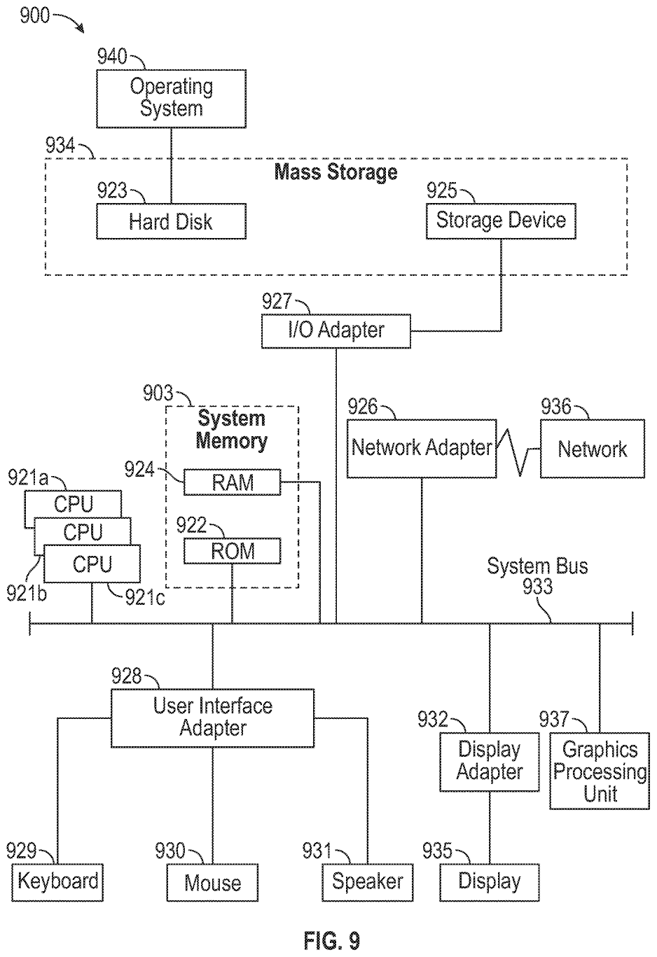

The computing system 102 includes one or more memory devices, one or more processors, a user interface device, among other components. All or a portion of the computing system 102 shown in FIG. 1 can be implemented for example, by all or a portion of computer system 900 of FIG. 9. Computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures, such as anatomical structures, surgical instruments 108 in the captured video of the surgical procedure. Features can further include events, such as phases and/or actions in the surgical procedure. Features that are detected can further include the actor 112 and/or patient 110. Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, vision transformers, encoders, decoders, or any other type of machine learning model. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video recording system 104. Alternatively, or in addition, the machine learning models use the surgical instrumentation data from the surgical instrumentation system 106. In yet other examples, the machine learning models use a combination of video data and surgical instrumentation data.

Additionally, in some examples, the machine learning models can also use audio data captured during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

In one or more examples, the machine learning models can detect surgical actions, surgical phases, anatomical structures, surgical instruments, and various other features from the data associated with a surgical procedure. The detection can be performed in real-time in some examples. Alternatively, or in addition, the computing system 102 analyzes the surgical data, i.e., the various types of data captured during the surgical procedure, in an offline manner (e.g., post-surgery). In one or more examples, the machine learning models detect surgical phases based on detecting some of the features, such as the anatomical structure, surgical instruments, and/or the like including combinations and/or multiples thereof. Offline analysis can be used to test the performance of future prediction accuracy and train models.

A data collection system 150 can be employed to store the surgical data, including the video(s) captured during the surgical procedures. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, and/or the like including combinations and/or multiples thereof. In some examples, the data collection system can use a distributed storage, i.e., the storage devices 152 are located at different geographic locations. The storage devices 152 can include any type of electronic data storage media used for recording machine-readable data, such as semiconductor-based, magnetic-based, optical-based storage media, and/or the like including combinations and/or multiples thereof. For example, the data storage media can include flash-based solid-state drives (SSDs), magnetic-based hard disk drives, magnetic tape, optical discs, and/or the like including combinations and/or multiples thereof.

In one or more examples, the data collection system 150 can be part of the video recording system 104, or vice-versa. In some examples, the data collection system 150, the video recording system 104, and the computing system 102, can communicate with each other via a communication network, which can be wired, wireless, or a combination thereof. The communication between the systems can include the transfer of data (e.g., video data, instrumentation data, and/or the like including combinations and/or multiples thereof), data manipulation commands (e.g., browse, copy, paste, move, delete, create, compress, and/or the like including combinations and/or multiples thereof), data manipulation results, and/or the like including combinations and/or multiples thereof. In one or more examples, the computing system 102 can manipulate the data already stored/being stored in the data collection system 150 based on outputs from the one or more machine learning models (e.g., phase detection, anatomical structure detection, surgical tool detection, and/or the like including combinations and/or multiples thereof). Alternatively, or in addition, the computing system 102 can manipulate the data already stored/being stored in the data collection system 150 based on information from the surgical instrumentation system 106.

In one or more examples, the video captured by the video recording system 104 is stored on the data collection system 150. In some examples, the computing system 102 curates parts of the video data being stored on the data collection system 150. In some examples, the computing system 102 filters the video captured by the video recording system 104 before it is stored on the data collection system 150. Alternatively, or in addition, the computing system 102 filters the video captured by the video recording system 104 after it is stored on the data collection system 150.

A 3D modeling system 120 can access one or more scanning devices 122 (e.g., computed tomography, magnetic resonance imaging, ultrasonic, and/or other such scanning devices) to scan and collect 3D image data of an anatomical feature of the patient 110. For example, the 3D modeling system 120 can construct and register a 3D model of a tumor or other features within the patient 110 to be used for predicting aspects during a surgical procedure, such as tumor location with respect to organs and one or more points of attachment. The 3D modeling system 120 can use various techniques to generate a 3D model and predict how and where the anatomical structure represented by the 3D model will appear in a future state of a surgical procedure that may later be observed by one or more of the cameras 105. For example, the 3D modeling system 120 can be a pre-operative system used to form a 3D model of a targeted aspect of the patient 110 prior to performing the surgical procedure.

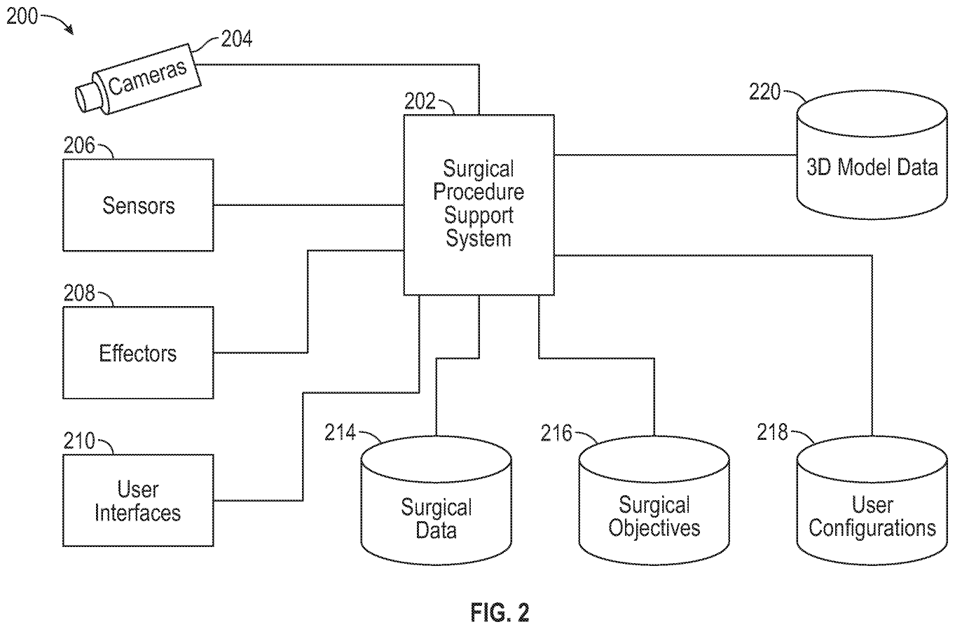

Turning now to FIG. 2, a surgical procedure system 200 is generally shown according to one or more aspects. The example of FIG. 2 depicts a surgical procedure support system 202 that can include or may be coupled to the CAS system 100 of FIG. 1. The surgical procedure support system 202 can acquire image or video data using one or more cameras 204. The surgical procedure support system 202 can also interface with one or more sensors 206 and/or one or more effectors 208. The sensors 206 may be associated with surgical support equipment and/or patient monitoring. The effectors 208 can be robotic components or other equipment controllable through the surgical procedure support system 202. The surgical procedure support system 202 can also interact with one or more user interfaces 210, such as various input and/or output devices. The surgical procedure support system 202 can store, access, and/or update surgical data 214 associated with a training dataset and/or live data as a surgical procedure is being performed on patient 110 of FIG. 1. The surgical procedure support system 202 can store, access, and/or update surgical objectives 216 to assist in training and guidance for one or more surgical procedures. User configurations 218 can track and store user preferences. The surgical procedure support system 202 can also access 3D model data 220 that may be populated by the 3D modeling system 120 of FIG. 1. The surgical procedure support system 202 can use the 3D model data 220 in making future state predictions during a surgical procedure as further described herein.

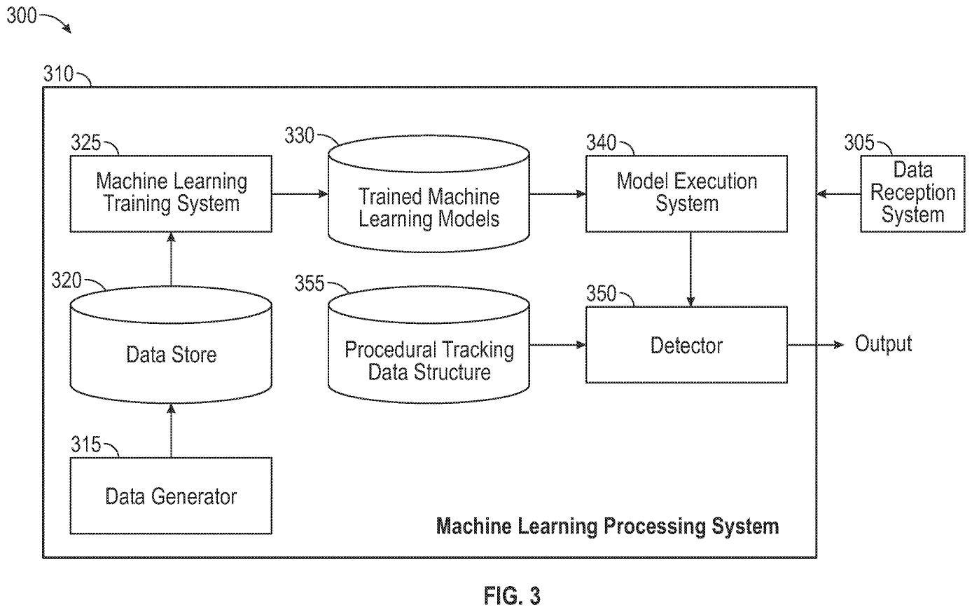

Turning now to FIG. 3, a system 300 for analyzing video and data is generally shown according to one or more aspects. In accordance with aspects, the video and data can be captured from video recording system 104 of FIG. 1. The analysis can result in predicting features that include surgical phases and structures (e.g., instruments, anatomical structures, and/or the like including combinations and/or multiples thereof) in the video data using machine learning, including predicting future states. System 300 can be the computing system 102 of FIG. 1, or a part thereof in one or more examples. System 300 uses data streams in the surgical data to identify procedural states according to some aspects.

System 300 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed. Alternatively, or in addition, the data reception system 305 can receive or access surgical data in an offline manner, for example, by accessing data that is stored in the data collection system 150 of FIG. 1.

System 300 further includes a machine learning processing system 310 that processes the surgical data using one or more machine learning models to identify one or more features, such as surgical phase, instrument, anatomical structure, critical structures, and/or the like including combinations and/or multiples thereof, in the surgical data. It will be appreciated that machine learning processing system 310 can include one or more devices (e.g., one or more servers), each of which can be configured to include part or all of one or more of the depicted components of the machine learning processing system 310. In some instances, a part or all of the machine learning processing system 310 is cloud-based and/or remote from an operating room and/or physical location corresponding to a part or all of data reception system 305. It will be appreciated that several components of the machine learning processing system 310 are depicted and described herein. However, the components are just one example structure of the machine learning processing system 310, and that in other examples, the machine learning processing system 310 can be structured using a different combination of the components. Such variations in the combination of the components are encompassed by the technical solutions described herein.

The machine learning processing system 310 includes a machine learning training system 325, which can be a separate device (e.g., server) that stores its output as one or more trained machine learning models 330. The machine learning models 330 are accessible by a model execution system 340. The model execution system 340 can be separate from the machine learning training system 325 in some examples. In other words, in some aspects, devices that “train” the models are separate from devices that “infer,” i.e., perform real-time processing of surgical data using the trained machine learning models 330.

Machine learning processing system 310, in some examples, further includes a data generator 315 to generate simulated surgical data, such as a set of synthetic images and/or synthetic video, in combination with real image and video data from the video recording system 104, to generate trained machine learning models 330. The data generator 315 can use the 3D model data 220 of FIG. 2 to generate predictive presentations to support training. Data generator 315 can access (read/write) a data store 320 to record data, including multiple models, images, and/or videos. The images and/or videos can include images and/or videos collected during one or more procedures (e.g., one or more surgical procedures). For example, the images and/or video may have been collected by a user device worn or used by the actor 112 of FIG. 1 (e.g., surgeon, surgical nurse, anesthesiologist, and/or the like including combinations and/or multiples thereof) during the surgery, a non-wearable imaging device located within an operating room, an endoscopic camera inserted inside the patient 110 of FIG. 1, and/or the like including combinations and/or multiples thereof. The data store 320 is separate from the data collection system 150 of FIG. 1 in some examples. In other examples, the data store 320 is part of the data collection system 150.

Each of the images and/or videos recorded in the data store 320 for performing training (e.g., generating the machine learning models 330) can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, and/or the like including combinations and/or multiples thereof). Further, the other data can include image-segmentation data that identifies and/or characterizes one or more objects (e.g., tools, anatomical objects, and/or the like including combinations and/or multiples thereof) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems. Timing based characterizations can also be learned to track median times in surgical phases or response times to events along with range bounds for faster and slower progressions relative to the median.

The machine learning training system 325 uses the recorded data in the data store 320, which can include the simulated surgical data (e.g., set of synthetic images and/or synthetic video based on 3D models) and/or actual surgical data to generate the trained machine learning models 330. The trained machine learning models 330 can be defined based on a type of model and a set of hyperparameters (e.g., defined based on input from a client device). The trained machine learning models 330 can be configured based on a set of parameters that can be dynamically defined based on (e.g., continuous or repeated) training (i.e., learning, parameter tuning). Machine learning training system 325 can use one or more optimization algorithms to define the set of parameters to minimize or maximize one or more loss functions. The set of (learned) parameters can be stored as part of the trained machine learning models 330 using a specific data structure for a particular trained machine learning model of the trained machine learning models 330. The data structure can also include one or more non-learnable variables (e.g., hyperparameters and/or model definitions).

As a further example, the machine learning training system 325 can train a spatio-temporal anticipative model (e.g., a spatio-temoporal predictor) using endoscope image frames and a computed tomography (CT) model deformably registered for each of the endoscope image frames. The spatio-temporal anticipative model can include a multiple layer neural network trained to predict the appearance of critical structures in different intervals of future times. The spatio-temporal anticipative model can also predict and visually illustrate/highlight the appearance of critical structures based on the CT model registration.

Model execution system 340 can access the data structure(s) of the trained machine learning models 330 and accordingly configure the trained machine learning models 330 for inference (e.g., prediction, classification, and/or the like including combinations and/or multiples thereof). The trained machine learning models 330 can include, for example, a fully convolutional network adaptation, an adversarial network model, an encoder, a decoder, or other types of machine learning models. The type of the trained machine learning models 330 can be indicated in the corresponding data structures. The trained machine learning models 330 can be configured in accordance with one or more hyperparameters and the set of learned parameters.

The trained machine learning models 330, during execution, receive, as input, surgical data to be processed and subsequently generate one or more inferences according to the training. For example, the video data captured by the video recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video recording system 104 can be received by the data reception system 305, which can include one or more devices located within an operating room where the surgical procedure is being performed. Alternatively, the data reception system 305 can include devices that are located remotely, to which the captured video data is streamed live during the performance of the surgical procedure. Alternatively, or in addition, the data reception system 305 accesses the data in an offline manner from the data collection system 150 or from any other data source (e.g., local or remote storage device).

The data reception system 305 can process the video and/or data received. The processing can include decoding when a video stream is received in an encoded format such that data for a sequence of images can be extracted and processed. The data reception system 305 can also process other types of data included in the input surgical data. For example, the surgical data can include additional data streams, such as audio data, RFID data, textual data, measurements from one or more surgical instruments/sensors, and/or the like including combinations and/or multiples thereof, that can represent stimuli/procedural states from the operating room. The data reception system 305 synchronizes the different inputs from the different devices/sensors before inputting them in the machine learning processing system 310.

The trained machine learning models 330, once trained, can analyze the input surgical data, and in one or more aspects, predict and/or characterize features (e.g., structures) included in the video data included with the surgical data. The video data can include sequential images and/or encoded video data (e.g., using digital video file/stream formats and/or codecs, such as MP4, MOV, AVI, WEBM, AVCHD, OGG, and/or the like including combinations and/or multiples thereof). The prediction and/or characterization of the features can include segmenting the video data or predicting the localization of the structures with a probabilistic heatmap. In some instances, the one or more trained machine learning models 330 include or are associated with a preprocessing or augmentation (e.g., intensity normalization, resizing, cropping, and/or the like including combinations and/or multiples thereof) that is performed prior to segmenting the video data. An output of the one or more trained machine learning models 330 can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The trained machine learning models 330, in one or more examples, are trained to perform higher-level predictions and tracking, such as predicting a phase of a surgical procedure and tracking one or more surgical instruments used in the surgical procedure. The coordinates can be projected with respect to the 3D model data 220 to determine current and future predicted locations of AI-identified features with the structure represented by the 3D model data 220.

While some techniques for predicting a surgical phase (“phase”) in the surgical procedure are described herein, it should be understood that any other technique for phase prediction can be used without affecting the aspects of the technical solutions described herein. In some examples, the machine learning processing system 310 includes a detector 350 that uses the trained machine learning models 330 to identify various items or states within the surgical procedure (“procedure”). The detector 350 can use a particular procedural tracking data structure 355 from a list of procedural tracking data structures. The detector 350 can select the procedural tracking data structure 355 based on the type of surgical procedure that is being performed. In one or more examples, the type of surgical procedure can be predetermined or input by actor 112. For instance, the procedural tracking data structure 355 can identify a set of potential phases that can correspond to a part of the specific type of procedure as “phase predictions”, where the detector 350 is a phase detector.

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, and/or the like including combinations and/or multiples thereof), pre-condition (e.g., lesions, polyps, and/or the like including combinations and/or multiples thereof). In some examples, the trained machine learning models 330 are trained to detect an “abnormal condition,” such as hemorrhaging, arrhythmias, blood vessel abnormality, and/or the like including combinations and/or multiples thereof.

Each node within the procedural tracking data structure 355 can identify one or more characteristics of the phase corresponding to that node. The characteristics can include visual characteristics. In some instances, the node identifies one or more tools that are typically in use or available for use (e.g., on a tool tray) during the phase. The node also identifies one or more roles of people who are typically performing a surgical task, a typical type of movement (e.g., of a hand or tool), and/or the like including combinations and/or multiples thereof. Thus, detector 350 can use the segmented data generated by model execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, and/or the like including combinations and/or multiples thereof).

The detector 350 can output predictions, such as a phase prediction associated with a portion of the video data that is analyzed by the machine learning processing system 310. The phase prediction is associated with the portion of the video data by identifying a start time and an end time of the portion of the video that is analyzed by the model execution system 340. The phase prediction that is output can include segments of the video where each segment corresponds to and includes an identity of a surgical phase as detected by the detector 350 based on the output of the model execution system 340. Further, the phase prediction, in one or more examples, can include additional data dimensions, such as, but not limited to, identities of the structures (e.g., instrument, anatomy, and/or the like including combinations and/or multiples thereof) that are identified by the model execution system 340 in the portion of the video that is analyzed. The phase prediction can also include a confidence score of the prediction. Other examples can include various other types of information in the phase prediction that is output. Further, other types of outputs of the detector 350 can include state information or other information used to generate audio output, visual output, and/or commands. For instance, the output can trigger an alert, an augmented visualization, identify a predicted current condition, identify a predicted future condition, command control of equipment, and/or result in other such data/commands being transmitted to a support system component, e.g., through surgical procedure support system 202 of FIG. 2.

It should be noted that although some of the drawings depict endoscopic videos being analyzed, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room (e.g., surgeon). Alternatively, or in addition, the cameras can be mounted on surgical instruments, walls, or other locations in the operating room. Alternatively, or in addition, the video can be images captured by other imaging modalities, such as ultrasound.

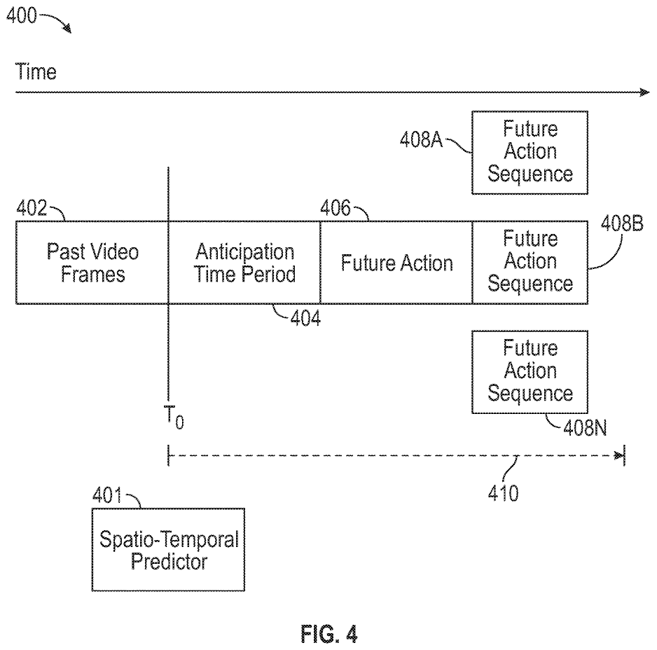

Turning now to FIG. 4, a time sequence diagram 400 of future action predictions is depicted according to one or more aspects. A spatio-temoporal predictor 401 can analyze a plurality of past video frames 402 of a surgical procedure up to a current point in time (T₀). The spatio-temoporal predictor 401 can use an anticipative model trained for the surgical procedure to look ahead through an anticipation time period 404 to predict one or more future actions 406 associated with the surgical procedure. The spatio-temoporal predictor 401 can also project further along a time continuum to predict one or more future action sequences 408A, 408B, 408C within a time window 410. The spatio-temoporal predictor 401 can use various sources of information to predict which of the future action sequences 408A, 408B, 408C has a higher likelihood of occurring, such as a current surgical phase, one or more next surgical phases, an observation of complications or deviation from an expected result previously observed during the surgical procedure, a level of complexity of a current and next sequence of actions to be performed, a pace of phase completion, and other such factors.

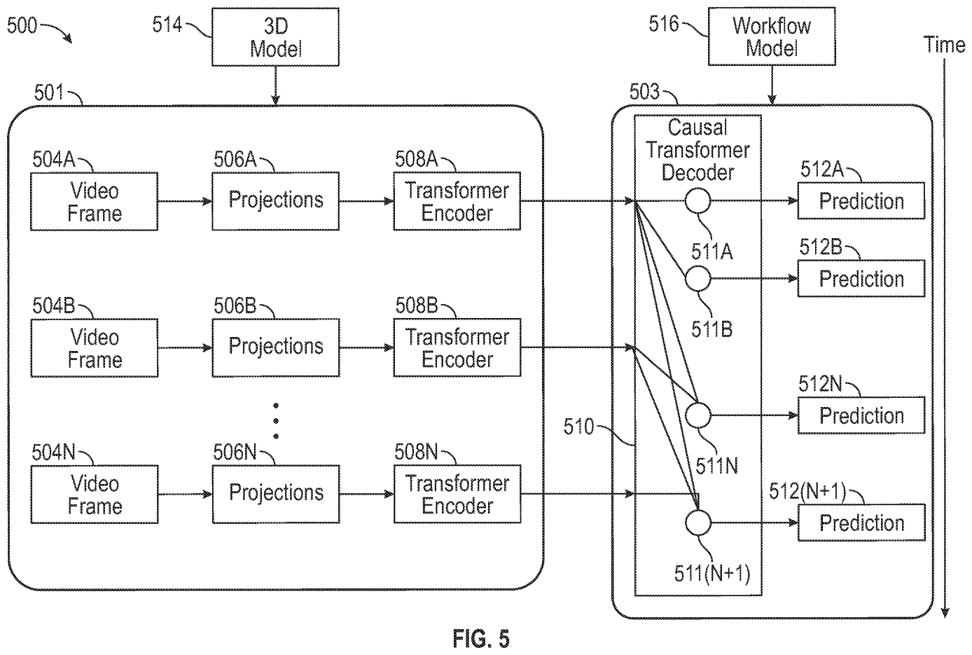

The spatio-temoporal predictor 401 can be part of the machine learning processing system 310 of FIG. 3, such as part of the model execution system 340 and/or detector 350, or may be implemented separately. As a further example, the spatio-temoporal predictor 401 can be part of or interface with the computing system 102 of FIG. 1 or the surgical procedure support system 202 of FIG. 2. As one example, the spatio-temoporal predictor 401 can implement an anticipative video transformer network, such as that depicted and described in the example of FIG. 5.

FIG. 5 depicts a block diagram of an anticipative video transformer network 500 according to one or more aspects. The anticipative video transformer network 500 can be an anticipative model implemented as a transformer model that splits a sequence of the video frames into non-overlapping patches in a feed-forward network that produces a plurality of predictions in a temporal attention portion based on a spatial-attention portion. A prediction can be selected from the plurality of predictions as a prediction output based on a prediction time window and a confidence score of the prediction. The prediction time window can be a variable range of time selected based on a prediction type performed. In the example of FIG. 5, the anticipative video transformer network 500 includes a backbone network 501 and a head network 503. Sequential video frames 504A, 504B, . . . , 504N of a surgical video captured by a camera (e.g., cameras 105, 204) can be decomposed into non-overlapping patches, e.g., 9 patches per frame, and linearly projected in a feature dimension as projections 506A, 506B, . . . , 506N respectively. The projections 506A, 506B, . . . , 506N can be provided to transformer encoders 508A, 508B, . . . , 508N respectively. The transformer encoders 508A, 508B, . . . , 508N can be attention-based frame encoders for spatial features in a single frame and for tracking across frames using tokens and weights before decoding. The backbone network 501 can also receive a 3D model 514 associated with the surgical procedure, for instance, from 3D model data 220 of FIG. 2. Spatial and appearance information from the 3D model 514 can be used to align with position and depth data in projections 506A-506N. Output of the backbone network 501 can be provided as input to the head network 503.

In the example of FIG. 5, the head network 503 includes a causal transformer decoder 510 with future feature predictors 511A, 511B, 511N, and 511(N+1). Predicted future features correspond to a frame feature in view of previous features. Predicted features can be decoded into a distribution over a semantic action class using a linear classifier. The causal transformer decoder 510 can be implemented as a masked transformer decoder by adding temporal position encoding to frame features using learned embedding of frame positions through one or more layers. Shared weights can be used across transformers applied to frames. Prediction outputs 512A, 512B, . . . , 512N, 512(N+1) can be future prediction states with tempo-spatial information defining possible future aspects with respect to a workflow model 516. The workflow model 516 can provide context for making predictions, such as surgical procedure, phase, expected phases sequences, and other such information. In the example of FIG. 5, the prediction output 512A aligns with a current time state, and prediction output 512B is a predicted future state determined based in part on video frame 504A without yet having knowledge of video frames 504B-504N. Prediction output 512N can be based on video frame features prior to video frame 504N, and prediction output 512(N+1) can be based on video frame features up to video frame 504N. Although depicted sequentially, the video frames 504A-504N need not occur in a back-to-back sequence but can represent time step differences (e.g., full second, half second, or quarter second intervals). Predictions can be classified as an event, an object position, and object appearance or other such future states. Predictions can include visual representations and/or notifications associated with a future state.

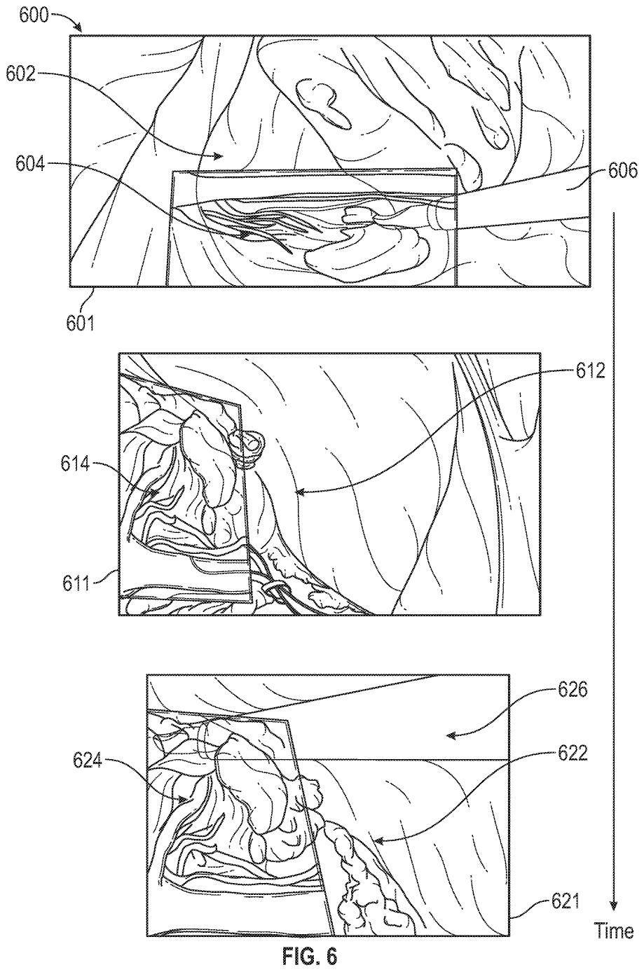

FIG. 6 depicts an image sequence 600 of critical structure prediction according to one or more aspects. In the example of FIG. 6, the spatio-temoporal predictor 401 of FIG. 4 can use the anticipative video transformer network 500 of FIG. 5 to predict where and how structures will appear in future states. For instance, in video frame 601, a 3D model 604 may be predicted as appearing behind an anatomical structure 602 as a surgeon moves a surgical instrument 606 in proximity to the anatomical structure 602. Notably, the actual structure represented by the 3D model 604 may not yet be exposed, such as prior to using the surgical instrument 606 to make an incision through the anatomical structure 602. Video frame 611 may depict a future time state relative to video frame 601. In video frame 611, a prediction output of the spatio-temoporal predictor 401 may predict that the structure represented by the 3D model 604 will appear as 3D model 614 with a different orientation and relative position as anatomical structure 612 is exposed. In video frame 621, the relative position and scale of structure represented by 3D model 614 in video frame 611 as 3D model 624 as the perspective of anatomical structure 622 shifts as compared to anatomical structure 612 and the addition of surgical instrument 626 moving into the video frame 621. The display of predicted future locations and appearance of the structure represented by 3D models 604, 614, and 624 can be selectable as a feature appearing in a user interface. A time window establishing how far into the future to generate and output predictions can be selectable. In some aspects, the 3D models 604, 614, and 624 can show a predicted current position where the structure represented by the 3D models 604, 614, and 624 is not currently visible. Future state predictions can be alerts that warn of predicted contact risk of the surgical instruments 606, 626 with the structure represented by the 3D models 604, 614, and 624 even though the structure represented by the 3D models 604, 614, and 624 has not yet become visible directly using a camera.

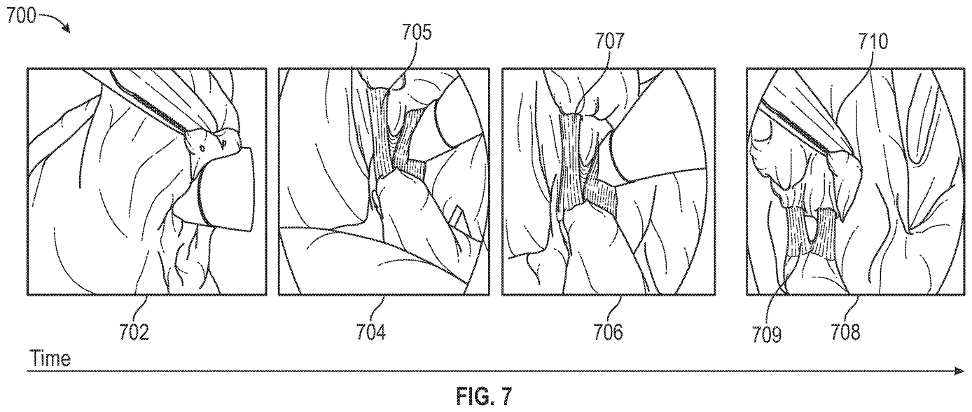

FIG. 7 depicts an image sequence 700 of temporal recognition of events according to one or more aspects. In the example of FIG. 7, a sequence of past video frames can be used by the spatio-temoporal predictor 401 of FIG. 4 up to a current video frame 702 to make a time sequence of future state predictions 704, 706, and 708. The spatio-temoporal predictor 401 of FIG. 4 can predict a first critical structure position 705 in future state prediction 704 and future changes as a second critical structure position 707 in future state prediction 706 and a third critical structure position 709 in future state prediction 708 to reach a final position 710 of a surgical instrument in the sequence. The amount of time predicted to elapse between the current video frame 702 and the future state predictions 704, 706, and 708 can be determined based on training that establishes a median pace and a range of expected times where no complications occur.

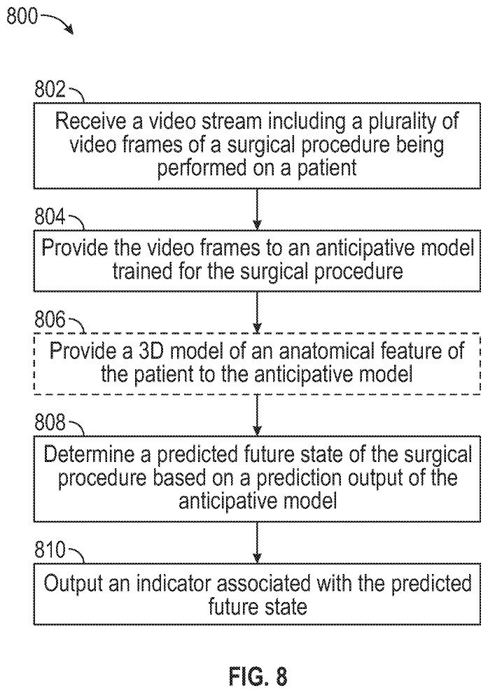

FIG. 8 depicts a flowchart of a method 800 of future state prediction of a surgical procedure according to one or more aspects. The method 800 can be executed by a system, such as system 300 of FIG. 3 as a computer-implemented method. For example, the method 800 can be implemented by spatio-temoporal predictor 401 of FIG. 4 within the model execution system 340 and/or detector 350 of FIG. 3. Further, the method 800 can be performed by a processing system, such as computer system 900 of FIG. 9.

At block 802, the spatio-temoporal predictor 401 can receive a video stream including a plurality of video frames of a surgical procedure, such as video frames 504A-504N of FIG. 5. At block 804, the spatio-temoporal predictor 401 can provide the video frames to an anticipative model trained for the surgical procedure, such as anticipative video transformer network 500. At block 806, the spatio-temoporal predictor 401 can provide a 3D model of an anatomical feature of the patient to the anticipative model, such as 3D model 514. At block 808, the spatio-temporal predictor 401 can determine a predicted future state of the surgical procedure based on a prediction output of the anticipative model. At block 810, the spatio-temoporal predictor 401 can output an indicator associated with the predicted future state.

According to some aspects, the method 800 can also include identifying a critical structure based on at least one of the video frames and predicting a future appearance of the critical structure as at least a portion of the predicted future state of the surgical procedure.

According to some aspects, the method 800 can include tracking a phase of the surgical procedure, where the future appearance of the critical structure is based at least in part on a current phase and one or more predicted future phases of the surgical procedure.

According to some aspects, the method 800 can include predicting coordinates of the critical structure relative to time as part of the predicted future state of the surgical procedure and predicting a location of an anatomical structure or surgical instrument relative to the coordinates of the critical structure.

According to some aspects, the indicator can include one or more of: a warning of a predicted issue with the surgical procedure, a notification of a predicted contact between a surgical instrument and the critical structure, and/or a notification of a predicted location of an anatomical structure not currently visible in the video frame.

According to some aspects, the anticipative model can be a transformer model that splits a sequence of the video frames into non-overlapping patches in a feed-forward network that produce a plurality of predictions in a temporal attention portion based on a spatial-attention portion.

According to some aspects, a prediction is selected from the plurality of predictions as the prediction output based on a prediction time window and a confidence score of the prediction.

According to some aspects, the prediction time window can be a variable range of time selected based on a prediction type performed.

It is understood that one or more aspects is capable of being implemented in conjunction with any other type of computing environment now known or later developed. For example, FIG. 9 depicts a block diagram of a computer system 900 for implementing the techniques described herein. In examples, computer system 900 has one or more central processing units (“processors” or “processing resources” or “processing devices”) 921a, 921b, 921c, etc. (collectively or generically referred to as processor(s) 921 and/or as processing device(s)). In aspects of the present disclosure, each processor 921 can include a reduced instruction set computer (RISC) microprocessor. Processors 921 are coupled to system memory 903 (e.g., random access memory (RAM) 924) and various other components via a system bus 933. Read only memory (ROM) 922 is coupled to system bus 933 and may include a basic input/output system (BIOS), which controls certain basic functions of computer system 900.

Further depicted are an input/output (I/O) adapter 927 and a network adapter 926 coupled to system bus 933. I/O adapter 927 may be a small computer system interface (SCSI) adapter that communicates with a hard disk 923 and/or a storage device 925 or any other similar component. I/O adapter 927, hard disk 923, and storage device 925 are collectively referred to herein as mass storage 934. Operating system 940 for execution on computer system 900 may be stored in mass storage 934. The network adapter 926 interconnects system bus 933 with an outside network 936 enabling computer system 900 to communicate with other such systems.

A display 935 (e.g., a display monitor) is connected to system bus 933 by display adapter 932, which may include a graphics adapter to improve the performance of graphics intensive applications and a video controller. In one aspect of the present disclosure, adapters 926, 927, and/or 932 may be connected to one or more I/O busses that are connected to system bus 933 via an intermediate bus bridge (not shown). Suitable I/O buses for connecting peripheral devices, such as hard disk controllers, network adapters, and graphics adapters typically include common protocols, such as the Peripheral Component Interconnect (PCI). Additional input/output devices are shown as connected to system bus 933 via user interface adapter 928 and display adapter 932. A keyboard 929, mouse 930, and speaker 931 may be interconnected to system bus 933 via user interface adapter 928, which may include, for example, a Super I/O chip integrating multiple device adapters into a single integrated circuit.

In some aspects of the present disclosure, computer system 900 includes a graphics processing unit 937. Graphics processing unit 937 is a specialized electronic circuit designed to manipulate and alter memory to accelerate the creation of images in a frame buffer intended for output to a display. In general, graphics processing unit 937 is very efficient at manipulating computer graphics and image processing, and has a highly parallel structure that makes it more effective than general-purpose CPUs for algorithms where processing of large blocks of data is done in parallel.

Thus, as configured herein, computer system 900 includes processing capability in the form of processors 921, storage capability including system memory (e.g., RAM 924), and mass storage 934, input means, such as keyboard 929 and mouse 930, and output capability including speaker 931 and display 935. In some aspects of the present disclosure, a portion of system memory (e.g., RAM 924) and mass storage 934 collectively store the operating system 940 to coordinate the functions of the various components shown in computer system 900.

It is to be understood that the block diagram of FIG. 9 is not intended to indicate that the computer system 900 is to include all of the components shown in FIG. 9. Rather, the computer system 900 can include any appropriate fewer or additional components not illustrated in FIG. 9 (e.g., additional memory components, embedded controllers, modules, additional network interfaces, etc.). Further, the aspects described herein with respect to computer system 900 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

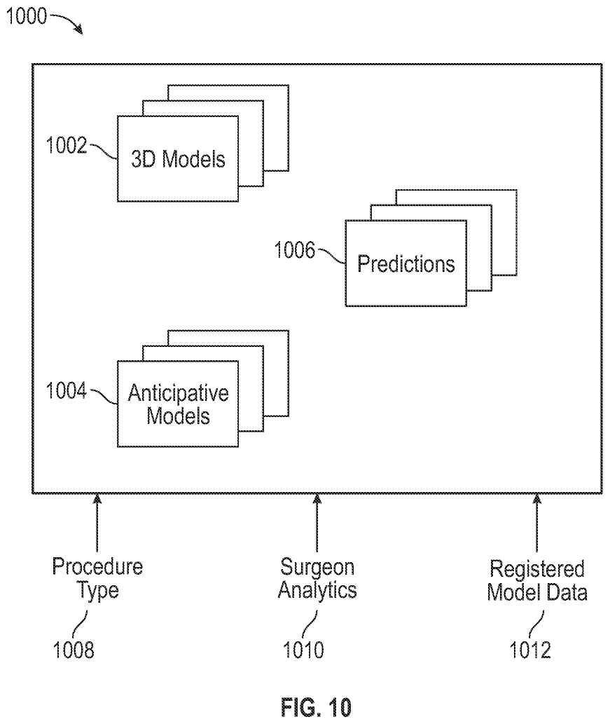

FIG. 10 depicts a system 1000 for predicting future states of surgical procedures according to one or more aspects. In the example of FIG. 10, multiple 3D models 1002 can be generated and used in combination with multiple anticipative models 1004 (e.g., spatio-temporal anticipative models) to generate multiple prediction outputs 1006. For example, 3D models 1002 can be generated for multiple patients using various techniques. The anticipative models 1004 can be trained to support multiple surgical procedural types. A procedure type 1008 can be provided during startup to select an appropriate one or more of the anticipative models 1004 and associated 3D models 1002. Surgeon analytics 1010 can be tracked as used as input to assist in tuning performance of the anticipative models 1004 and accuracy of the prediction outputs 1006. As one example, a degree of alignment between a current state and a previously predicted future state can be determined, and a surgeon assessment score based on the degree of alignment can be generated as part of the surgeon analytics 1010. The surgeon analytics 1010 can also include trend data to identify how closely surgeon performance aligns with previous observations and/or models. Registered model data 1012 can be collected from another source, such as 3D modeling system 120 of FIG. 1 to train the 3D models 1002 using, for example, machine learning processing system 310 of FIG. 3. For instance, self-supervised learning can be used to capitalize on a large corpus of unlabeled videos. The anticipative models 1004 can be trained on a per procedure basis. Transformers can be used for end-to-end attention-based video modeling, for instance, using the anticipative video transformer network 500 to train the anticipative models 1004 for various procedure types. Training of the anticipative models 1004 can include learning a pace of procedure corelated to a plurality of phases of the surgical procedure.

Intermediate future prediction losses can be used during training for predictive video representation. For instance, a next surgical step prediction can be supervised using cross-entropy loss for labeled future surgical steps. Cross-modal features can be supervised for appearance prediction loss. Dense visual appearance prediction can be used, for example, where endoscopic visual images are available for a sequence of frames under analysis. Depth map predictions can be based on stereo or monocular depth estimations, for instance, where depth information is available. Where a pre-operative 3D model is available, a sparse deformed 3D model can be registered for predictions. Incorporation of intermediate future prediction losses can encourage a predictive surgical video representation that picks up patterns in how visual activity is likely to unfold in one or more future states.

Examples of anticipation can include that a mistake is about to be made, illustrating where and when a critical structure will appear, predicting a future state of a structure represented by the 3D models 1002. As an example a model may first encode depth and visual features from initial look phase, move on to fascia being removed from kidney, and finally predict the next action will be dissection of the kidney where a tumor is registered using pre-op CT deformable registration in the registered model data 1012.

Temporal structure in weakly-labeled surgical videos can be used to train deep networks to predict visual representation of images in future. The spatio-temoporal predictor 401 can anticipate both actions and objects up to a few seconds to a few minutes into the future, for example. Given an input frame, the anticipative models 1004 can be trained to predict multiple representations in the future that can each be classified into actions. When the future is uncertain, each network can predict a different representation, allowing for multiple action forecasts. To obtain the most likely future action, distributions can be marginalized from each network. Technical effects can include future prediction of critical structures and other objects/events in a surgical procedure.

According to an aspect, a system can include a machine learning training system configured to use a training dataset to train a 3D model of an anatomical structure and an anticipative model to predict a future state of a surgical procedure based on the training dataset and the 3D model. The system can also include a data collection system configured to capture a video of the surgical procedure and a model execution system configured to execute the anticipative model to determine a predicted future state of the surgical procedure based on a prediction output of the anticipative model with respect to the video. The system can further include a detector configured to generate an indicator associated with the predicted future state and output the indicator to a user interface.

According to some aspects, the 3D model can be based on image data collected using one or more scanning devices to scan a patient upon which the surgical procedure is performed.

According to some aspects, the anticipative model can be trained on a per procedure basis using self-supervised learning and end-to-end attention-based video modeling.

According to some aspects, the machine learning training system can be configured to train the anticipative model to learn a pace of procedure corelated to a plurality of phases of the surgical procedure, where a time window used to determine the predicted future state is adjusted based on comparing an observed pace to the pace of procedure as learned during training.

According to some aspects, the anticipative model can be trained based on a plurality of endoscope image frames and a computed tomography model deformably registered for each of the endoscope image frames to predict an appearance of one or more critical structures at one or more future times in different intervals.

According to some aspects, the predicted future state of the surgical procedure can include one or more anticipated actions and object states.

According to an aspect, a computer program product can include a memory device having computer executable instructions stored thereon, which when executed by one or more processors cause the one or more processors to perform a plurality of operations. The operations can include receiving a video stream including a plurality of video frames of a surgical procedure, providing the video frames to an anticipative model trained for the surgical procedure, determining a predicted future state of the surgical procedure based on a prediction output of the anticipative model, and outputting an indicator associated with the predicted future state.

According to some aspects, the operations can include identifying a critical structure based on at least one of the video frames and predicting a future appearance and/or a future location of the critical structure as at least a portion of the predicted future state of the surgical procedure.

According to some aspects, the operations can include tracking a phase of the surgical procedure, where the predicted future state is based at least in part on a current phase and one or more predicted future phases of the surgical procedure.

According to some aspects, the operations can include determining a degree of alignment between a current state and a previously predicted future state, and generating a surgeon assessment score based on the degree of alignment.

According to some aspects, the operations can include tracking the surgeon assessment score over a predetermined period of time to determine a current trend of the surgery, and selecting the predicted future state of the surgical procedure from a plurality of prediction outputs that more closely aligns with the current trend of the surgery.

According to some aspects, the predicted future state can include a visual representation of one or more images at one or more future time steps.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language, such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium, such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A computer-implemented method comprising:
receiving a video stream comprising a plurality of video frames of a surgical procedure;
providing the video frames to an anticipative model trained for the surgical procedure;
providing a three-dimensional (3D) model of an anatomical feature of the patient to the anticipative model;
determining a predicted future state of the surgical procedure based on a prediction output of the anticipative model; and
outputting an indicator associated with the predicted future state.

2. The computer-implemented method of claim 1, further comprising:
identifying a critical structure based on at least one of the video frames; and
predicting a future appearance of the critical structure as at least a portion of the predicted future state of the surgical procedure.

3. The computer-implemented method of claim 2, further comprising:
tracking a phase of the surgical procedure, wherein the future appearance of the critical structure is based at least in part on a current phase and one or more predicted future phases of the surgical procedure.

4. The computer-implemented method of claim 2, further comprising:
predicting coordinates of the critical structure relative to time as part of the predicted future state of the surgical procedure; and
predicting a location of an anatomical structure or surgical instrument relative to the coordinates of the critical structure.

5. The computer-implemented method of claim 1, wherein the indicator comprises one or more of: a warning of a predicted issue with the surgical procedure, a notification of a predicted contact between a surgical instrument and the critical structure, and/or a notification of a predicted location of an anatomical structure not currently visible in the video frame.

6. The computer-implemented method of claim 1, wherein the anticipative model is a transformer model that splits a sequence of the video frames into non-overlapping patches in a feed-forward network that produce a plurality of predictions in a temporal attention portion based on a spatial-attention portion.

7. The computer-implemented method of claim 6, wherein a prediction is selected from the plurality of predictions as the prediction output based on a prediction time window and a confidence score of the prediction.

8. The computer-implemented method of claim 7, wherein the prediction time window is a variable range of time selected based on a prediction type performed.

9. A system comprising:
a machine learning training system configured to use a training dataset to train:
a three-dimensional (3D) model of an anatomical structure; and
an anticipative model to predict a future state of a surgical procedure based on the training dataset and the 3D model;
a data collection system configured to capture a video of the surgical procedure;
a model execution system configured to execute the anticipative model to determine a predicted future state of the surgical procedure based on a prediction output of the anticipative model with respect to the video; and
a detector configured to:
generate an indicator associated with the predicted future state; and
output the indicator to a user interface.

10. The system of claim 9, wherein the 3D model is based on image data collected using one or more scanning devices to scan a patient upon which the surgical procedure is performed.

11. The system of claim 9, wherein the anticipative model is trained on a per procedure basis using self-supervised learning and end-to-end attention-based video modeling.

12. The system of claim 9, wherein the machine learning training system is configured to:
train the anticipative model to learn a pace of procedure corelated to a plurality of phases of the surgical procedure, wherein a time window used to determine the predicted future state is adjusted based on comparing an observed pace to the pace of procedure as learned during training.

13. The system of claim 9, wherein the anticipative model is trained based on a plurality of endoscope image frames and a computed tomography model deformably registered for each of the endoscope image frames to predict an appearance of one or more critical structures at one or more future times in different intervals.

14. The system of claim 9, wherein the predicted future state of the surgical procedure comprises one or more anticipated actions and object states.

15. A computer program product comprising a memory device having computer executable instructions stored thereon, which when executed by one or more processors cause the one or more processors to perform a plurality of operations comprising:
receiving a video stream comprising a plurality of video frames of a surgical procedure;
providing the video frames to an anticipative model trained for the surgical procedure;
determining a predicted future state of the surgical procedure based on a prediction output of the anticipative model; and
outputting an indicator associated with the predicted future state.

16. The computer program product of claim 15, wherein the operations further comprise:
identifying a critical structure based on at least one of the video frames; and
predicting a future appearance and/or a future location of the critical structure as at least a portion of the predicted future state of the surgical procedure.

17. The computer program product of claim 15, wherein the operations further comprise:
tracking a phase of the surgical procedure, wherein the predicted future state is based at least in part on a current phase and one or more predicted future phases of the surgical procedure.

18. The computer program product of claim 15, wherein the operations further comprise:
determining a degree of alignment between a current state and a previously predicted future state; and
generating a surgeon assessment score based on the degree of alignment.

19. The computer program product of claim 18, wherein the operations further comprise:
tracking the surgeon assessment score over a predetermined period of time to determine a current trend of the surgery; and
selecting the predicted future state of the surgical procedure from a plurality of prediction outputs that more closely aligns with the current trend of the surgery.

20. The computer program product of claim 15, wherein the predicted future state comprises a visual representation of one or more images at one or more future time steps.
